(19) United States
(12) Patent Application Publication
(10) Pub. No.: US 2004/0131567 A1
(43) Pub. Date: Jul. 8, 2004

(54) Antibacterial topical formulations

(76) Inventor: Joe S. Wilkins JR., Galveston, TX (US)

Correspondence Address: Laura G. Barrow, Esq., P.O. Box 215, Estero, FL, 33928-0215, US

(21) Appl. No.: 10/615,589

(22) Filed: Jul. 8, 2003

Related U.S. Application Data
(60) Provisional application No. 60/394,333, filed on Jul. 8, 2002.

(51) Int. Cl. A61K 7/06; A61K 31/01

(52) U.S. Cl. 424/70.1; 514/762

(57) ABSTRACT

Novel topical formulations and methods use are described herein for eradicating various disease-causing pathogens. The inventive formulations comprise effective amounts of d-limonene.

This is application claims the benefit of the filing of co-pending U.S. provisional application serial No. 60/394,333 filed Jul. 8, 2002, which is incorporated by reference herein in its entirety.

### BACKGROUND AND SUMMARY OF THE INVENTION:

Limonene is a monocyclic monoterpene commonly found in the form of its d-isomer. d-limonene is one of the most common terpenes in nature, occurring in citrus and a wide variety of other plant species.

The present invention is directed to topical formulations, including dermatological and nasal inhalent formulations. In particular, the formulations comprise, in part, limonene as an active ingredient in killing or inhibiting the growth of a variety of bacterial pathogens known to cause a number of infectious diseases in humans and animals. Specifically, in vitro analyses revealed that d-limonene is effective in eradicating the following major gram-positive pathogens: Staphylococcus aureus, Staphylococcus epidermidis (both methicillin sensitive and resistant), Streptococcus pyogenes, Streptococcus mutans, and other beta hemolytic streptococci, Entercoccus faecalis, and Enterococcus faecium (both vancomycin sensitive and resistant). In vitro tests further revealed that d-limonene is effective in eradicating the following gram-negative pathogens: Escherichia coli, Enterobactor cloacae, Klebsiella pneumoniae, Serratia marcescens, Pseudomonas aeruginosa, Acinetobacter baummii/haemolyticus, Paenibacillus polymyxa, and Stenotrophomonas maltophilia. In vitro tests also revealed that d-limonene is effective in eradicating various Bacillus species, such as Bacillus licheniformis, B. sphaericus, Bacillus cereus, Bacillus subtilus, including the species strain of anthrax (Bacillus anthracis—Stearns and Ames strains). The microbial assay methodology, and results, are described in Example 1 and Tables 1-2.

In view of the in vitro anti-microbial activity of d-limonene, the present invention is directed to formulations and methods of using these formulations for treating a variety of systemic and local bacterial skin infections in humans and animals, including topical dermatological preparations comprising d-limonene. Such preparations have been shown effective in treating dermatological infections as well as eczema and psoriasis.

### DETAILED DESCRIPTION OF THE PREFERRED EMBODIMENTS

The present invention is directed to dermatological compositions for killing or inhibiting the growth a variety of bacterial pathogens known to cause a number of infectious diseases in humans and animals. As used herein, the term “animal” shall include humans as well as non-human animals, namely mammals, reptiles, and birds. Specifically, the present invention is directed to dermatological compositions comprising limonene for use in killing or inhibiting the growth of bacteria responsible for causing various localized dermatological infections (i.e. Streptococcus and Staphylococcus species) as well as psoriasis and eczema.

In certain embodiments, the formulations comprise an effective amount of d-limonene, preferably from about 10% to about 50% d-limonene mixed in a compatible vehicle, such as Vitamin E oil. A preferred formulation comprises only d-limonene and Vitamin E; however, it will be recognized by those of ordinary skill in the art at that other pharmaceutical bases conventionally used in the formulation of topical ointments, lotions, creams, solutions, shampoos, body soap, and the like may be employed. When d-limonene is combined with Vitamin E, the composition is heated to about 100° F. for a sufficient time during blending until the d-limonene is completely mixed therein (i.e. until a substantially homogenous mixture results).

The inventive topical composition may be formulated in the aforementioned Vitamin E solution or various types of ointments, creams, lotions, and the like, and then applied to the affected area on the patient's skin. When an effective amount of the inventive composition is applied to the affected area on the patients skin, healing effects are observed in less than a week. In a Vitamin E solution comprising about 30% d-limonene, application of about 1 ml to 2 ml of the solution to an arm affected with psoriasis, the psoriasis was relieved within 72 hours after the first application. In addition, skin infections present and resulting from the dry and broken skin caused by the psoriasis condition were also healed within the same 72-hour period.

As described in more detail in the following examples, d-limonene, and in particular highly purified d-limonene (i.e. at least 98.5% purity), has been shown to be effective in killing or inhibiting the growth of a number of gram-positive and gram-negative bacteria, including Staphylococcus aureus and epidermidis (both methicillin-sensitive and resistant), Enterococcus faecalis and faecium, Escherichia coli, Enterobacter cloacae, Klebsiella pneumoniae, Serratia marcescens, Pseudomonas aeruginosa, Acinetobacter baummii/haemolytics and Stenotrophomonas maltophila. D-limonene has also proven effective in eradicating various strains of Bacillus, including the Stearns and Ames strain of Bacillus anthracis, Bacillus licheniformis, Bacillus subtilis, Bacillus sphaericus, Bacillus cereus, and Paenibacillus polymyxa. These results indicate that in addition to use as a dermatological anti-infective, for example, effective amounts of d-limonene may be formulated in an nasal solution for spray or drop instillation for the treatment or prevention of infection within the nasal and sinus cavity. These results also suggest effectiveness of formulating d-limonene in an oral inhalant for inspiration-within the lungs and bronchial airways for the eradication of airborne bacteria, including the Bacillus species responsible for causing anthrax.

The d-limonene may be purified by known distillation techniques, such as that described in U.S. Pat. No. 6,420,435, which is incorporated herein by reference in its entirety.

### EXAMPLE 1

Clinical isolates (10⁵ bacteria/ml) (about 100 μl) of gram-positive pathogens (Staphylococcus aureus and epidermidis (both methicillin-sensitive and resistant) plus Enterococcus faecalis and faecium) along with a group of gram-negative pathogens (Escherichia coli, Enterobacter cloacae, Klebsiella pneumoniae and Serratia marcescens coupled with opportunistic pathogens Pseudomonas aeruginosa, Acinetobacter baummii/haemolyticus and Stenotrophomonas maltophila) were each inoculated into 2 ml of d-limonene, in accordance with the standard phenol-coefficient assay and other screening methodology for plant antimicrobial activity and incubated for 72 hrs. A 2 ml broth media was used as a positive control. The d-limonene used was purified to at least 98.5% via a distillation process. The product was purified and examined for purity via HPLC.

Aliquots were subsequently cultured at 24 hours, 48 hours, and 72 hours to determine the antimicrobial effect. Appropriate media was inoculated in accordance with NCCLS standards. Blood agar was used for the gram-positive organisms, while McConkey Agar was utilized for the gram-negative organisms. ATCC strains of S. aureus, E. faecalis, P. aeruginosa, and E. coli were used as controls organisms and compared to the clinical isolates of these pathogens.

The results of the assay are shown in Table 1 (gram-positive organisms) and Table 2 (gram-negative organisms), wherein all of the pathogens tested were shown to be effectively eradicated within 24 hours.

Cultures were held 72 hours to ascertain if a resistant genetic code might have been facilitated. The response to subculture at 72 hours yielded no-growth, thus clearly indicating that no mutagenic or plasmid transposon was noted.

TABLE 1
Antibacterial effects of d-limonene on gram-positive organisms

| | Concentrations | Growth at | | |
|---|---|---|---|---|
| Organism | CFU/ml | 24 hr | 48 hr | 72 hr |
| S. aureus | >10⁵ | NG* | NG | NG |
| S. epidermidis | >10⁵ | NG | NG | NG |
| E. faecalis | >10⁵ | NG | NG | NG |
| E. faecium | >10⁵ | NG | NG | NG |

*NG = no growth

TABLE 2
Antibacterial effects of d-limonene on gram-negative organisms

| | Concentrations | Growth at | | |
|---|---|---|---|---|
| Organism | CFU/ml | 24 hr | 48 hr | 72 hr |
| E. coli | >10⁵ | NG | NG | NG |
| Ent. cloacae | >10⁵ | NG | NG | NG |
| K. pneumoniae | >10⁵ | NG | NG | NG |
| S. marcescens | >10⁵ | NG* | NG | NG |
| P. aeruginosa | >10⁵ | NG | NG | NG |
| Ac. baum/haemo | >10⁵ | NG | NG | NG |
| S. maltophilia | >10⁵ | NG | NG | NG |

*NG = no growth

### EXAMPLE 2

A topical composition was manufactured by combining the following components:

30% d-limonene

70% Vitamin E oil

The two components were blended, while heating (up to 100° F.) for about 15 minutes until the homogenous.

### EXAMPLE 3

The Stearns and Ames strain of Bacillus anthracis were subjected to a battery of standard topical anti-bacterials, nutriceuticals, and herbals, including SILVADENE (generic silver sulfadiazine, vended by Hoescht Marion Roussel, now Par); SILVADENE with nystatin 0.025%; mafenide acetate, FURACIN (generic nitrofurazone, vended by Roberts), bacitracin with Polymyxin B (Poly B), silver nitrate, sodium hypochlorite (NaOCI), grapefruit seed extract (GSE), oleander extract with Aloe vera (Biotonics, San Antonio, Tex.) and FX (vended by Sterifx, Inc, Shreveport, La.). Both B. anthracis strains were tested by Nathans Agar Well Diffusion Technique.

Results: The Stearns strain of B. anthracis was susceptible to all products tested except Bacitracin, Poly B and NaOCl. The most effective among the standard topicals was Bactroban® with an average inhibition zone of 45 mm, followed by mafenide acetate at 38 mm. Furacin was 33 mm with Silvadene at 19 mm. Both Silvadene with Nystatin and AgNO3 zones of inhibition were 18 mm. The nutraceuticals GSE and d-limonene had zones of inhibition of 25 mm and 30 mm, respectively, whereas the Oleander with Aloe vera had a zone size of 20 mm. The Fx product at 1× had no zone of inhibition while the 4× and 12× zones were 25 mm and 32 mm, respectively. The zones of inhibition for the more lethal and pathogenic Ames strain were comparable to those of the Stearns strain for the standard anti-infectives, nutraceuticals (i.e. GSE and d-limonene) and herbal products. Again, mafenide acetate and Bactrobans were at the top of the susceptibility list at 34 mm vs 35 mm, respectively as was the Fx 4× and 12× both at 35 mm and 46 mm, respectively. GSE and Fx 1× zones of inhibition were both at 23 mm. The zone of inhibition for d-limonene was 21 mm. SILVADENE was at 18 mm while Nystatin/SILVADENE was 14 mm. AgNO₃ zones of inhibition was at 16 mm as was the Oleander Aloe vera product. Bacitracin, Polymyxin B and NaOCl were ineffective showing no zones of inhibition. Both strains of B. anthracis were susceptible to the standard topical antimicrobials. Bactroban®, mafenide acetate and Silvadene®. The commercial Fx product was very effective at 4× and 12× concentrations. The majority of products tested inhibited the growth of both strains of B. anthracis.

### EXAMPLE 4

Methods: Six strains of Bacillus species were tested using the Nathans Agar Well Diffusion technique in 3 replicate assays. The strain included ATCC strains of Paenibacillus polymyxa, Bacillus licheniformis, Bacillus subtilis, Bacillus sphaericus, Bacillus cereus and a wild Bacillus strain from a burn patient. The anti-infectives tested were SILVADENE, mafenide acetate, Furacin, BACTROBAN, Bacitracin plus Polymyxin B, SILVADENE with Nystatin, 0.025% NaOCl, AgNO₃, Grapefruit Seed Extract (GSE), d-limonene, Oleander extract with Aloe vera and various concentrations of a new anti-infective solution.

Results: All anti-infectives tested were effective against all strains of Bacillus except Bacitracin with Polymyxin B where none of the strains were inhibited and NaOCl were only inhibited P. Polymyxa, B. sphaericus and B. cereus with an average zone size of 16 mm. BACTROBAN's average zone of inhibition was 46 mm followed by mafenide acetate at 36 mm. Furacin was 35 mm, Silvadene was 26 mm, followed by GSE at 25 mm. SILVADENE with Nystatin was 24 mm, while Fx 1× (Sterifx, Inc, Shreveport, La.) was only effective against B. subtilis, B. sphaericus and P. polymyxa at 22 mm. Fx5× and Fx10× inhibited all Bacillus strains tested with an average zone size of 32 mm and 49 mm respectively. The Oleander extract was 18 mm while d-limonene zones were 21 mm and AgNO3 was 16 mm.

Conclusions: The standard topicals used in soft tissue wound infections could effectively eradicate cutaneous B. anthracis as would the nutriceuticals (i.e. d-limonene) and herbals tested. Moreover, the herbals and nutraeuticals could be employed effectively as aerosols in the case of inhalation anthrax, and thus, could effectively be used as therapeutic alternatives for B. anthracis infections.

### EXAMPLE 5

The topical composition recited in Example 2 was used to treat a 59-year old male suffering from psoriasis on his hands. The male subject also suffered from localized minor skin infections due to extremely dry skin resulting from the psoriasis. The male subject had in the past tried treating his condition with Vitamin E alone, with no results. The subject has also tried using the 20 mg SORIATANE and CIPRO. Neither treatment was effective in alleviating the psoriasis. Moreover, the subject experienced unpleasant side effects with the prescription regimen.

About 1 ml of the topical composition recited in Example 2 was applied to the affected area on the subject's hands, twice daily, for at least 72 hours. After three days, the pruritis, pain, and inflammation of the psoriasis were no longer observed or experienced, and the skin infections began to heal during this time period, as well (i.e. no signs of infection were observed or pain experienced).

### EXAMPLE 6

The inventor, a 54-year old male, applied the composition recited in Example 2 to his dry/cracking and scaly elbows twice a day for 3 days. After 3 days of therapy, the erythema and pruitis were relieved, with the skin returned to a normal color and with normal skin characteristics (e.g. no more scales). Prior to treatment with the inventive composition, the inventor had tried a regimen of Vitamin E alone, with no improvement in his condition.

I claim:

1. A method for killing or inhibiting the growth of bacteria externally on the skin or within the nasal cavity of an animal, said method comprising administering a therapeutically effective amount of a formulation comprising d-limonene to the animal's skin or nasal cavity for a time sufficient to effectively eradicate or inhibit the growth of said bacteria.

2. The method of claim 1, wherein said d-limonene has a purity of at least 98.5%.

3. The method of claim 1, wherein said bacteria are selected from the group of Staphylococcus aureus and epidermidis, Enterococcus faecalis and faecium, Escherichia coli, Enterobacter cloacae, Strep. pyogenes, Klebsiella pneumoniae, Serratia marcescens, Pseudomonas aeruginosa, Acinetobacter baummii/haemolytics and Stenotrophomonas maltophila, Bacillus anthracis, Bacillus licheniformis, Bacillus subtilis, Bacillus sphaericus, Bacillus cereus, and Paenibacillus polymyxa.

4. The method of claim 3, wherein said d-limonene has a purity of at least 98.5%.

5. The method of claim 1, wherein said formulation is selected from the group of Vitamin E oil, lotions, ointments, solutions, suspensions, creams, shampoos, and soaps.

6. A method for treating psoriasis in an animal, said method comprising applying a topical formulation to an area affected with psoriasis for a time sufficient to effectively erradicate said psorasis, said formulation comprising a therapeutically effective amount of d-limonene.

7. The method of claim 6, wherein said d-limonene has a purity of at least 98.5%.

8. The method of claim 6, wherein said formulation comprises from about 10% to about 50% d-limonene.

9. The method of claim 8, wherein said d-limonene has a purity of at least 98.5%.

10. The method of claim 6, wherein said formulation comprises about 20-30% d-limonene.

11. The method of claim 10, wherein said d-limonene has a purity of at least 98.5%.

12. A topical dermatological formulation comprising d-limonene and Vitamin E oil.

13. The formulation of claim 12, wherein said d-limonene comprises from about 10% to about 50% of said formulation.

14. The formulation of claim 12, wherein said d-limonene has a purity of at least 98.5%.

15. The formulation of claim 13, wherein said d-limonene comprises from about 20% to about 30% of said formulation.

16. The formulation of claim 15, wherein said d-limonene has a purity of at least 98.5%.

17. The formulation of claim 13, wherein said d-limonene has a purity of at least 98.5%.
